(19) United States
(12) Patent Application Publication
(10) Pub. No.: US 2025/0049577 A1
(43) Pub. Date: Feb. 13, 2025

(54) SYSTEM FOR PERFORMING POSTERIOR SPINAL ARTHRODESIS AT A FACET JOINT

(71) Applicant: SC MEDICA, Strasbourg (FR)

(72) Inventor: Camille SROUR, Strasbourg (FR)

(21) Appl. No.: 18/798,524

(22) Filed: Aug. 8, 2024

(30) Foreign Application Priority Data
Aug. 10, 2023 (FR) 2308638

(51) Int. Cl. A61F 2/44; A61F 2/30

(52) U.S. Cl. CPC A61F 2/4405; A61F 2002/30112

(57) ABSTRACT

The implant device for performing posterior spinal arthrodesis at a facet joint is in the form of a flat element of constant thickness. The element has reliefs on each of its faces so as to form an anchor associated with at least one complementary anchor, such as a screw with a diameter greater than the thickness of the element. The complementary anchor can be screwed into the flat element such that at least some of its threads protrude from the faces. The element has a U-shape with a transverse part from which two branches extend. On the proximal side, the free ends of the branches have retention devices, on the inner side of the U so as to permit the screwing and insertion of the screw between the branches.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to a system for performing posterior spinal arthrodesis at a facet joint, said system comprising an implant device and its retaining means.

### 2. Description of Related Art Including Information Disclosed Under 37 CFR 1.97 and 37 CFR 1.98

Such a posterior procedure immobilizes two vertebrae by fusing each of the lower articular processes of one vertebra with the upper articular process of the neighboring lower vertebra by inserting an implant device of the cage type containing a bone graft into the interface space.

In practice, the surgeon prepares the implant site by removing the joint capsule with an instrument called a facet chisel, then uses a rasp to “bleed” the parts opposite the joint space on the facets. This step with the rasp has two purposes: to prepare the pre-existing space, with a boneless but often slightly curved interline, for the straight shape of the implant, and to promote subsequent fusion of the facets through the implant that has bled/revived the facets down to the cancellous bone.

Implant devices for this purpose are already known, consisting of a wedge-shaped element whose two opposite faces, each intended to come into contact with one facet, have a surface provided with reliefs to ensure anchorage, and which is pierced with a multiplicity of holes intended for the development of bone material after deposition of a bone graft. However, the wedge shape, despite the reliefs, tends not to prevent expulsion, so these implant devices are associated with at least one screw, passing through said implant device via a hole and screwed into a process obliquely relative to the median plane of the implant. WO 2012154653 describes such an implant device, fitted with two screws. However, the low degree of inclination of the screw axes means that they are practically parallel to said median plane, so that there is little bone material between the screws and the veneer walls, which are then weakened, making the fastening less certain.

Already known from document WO2019016341 to the present Applicant is an implant in the shape of a flat element of constant thickness, comprising, in order to receive a bone graft, a large central opening which gives it a substantially annular shape, and which device is intended to be inserted into a cavity previously made at the location of the facet joint to be immobilized; said element being D-shaped and comprises a rounded side which constitutes the distal edge for being inserted into said cavity, while the opposite straight proximal edge comprises a means for securing to a holding and positioning tool; while the opposite surfaces of said flat element comprise reliefs which consist of reliefs arranged perpendicularly to the insertion direction, each of the reliefs having a tooth profile with two faces of which one, the face oriented on the side of said proximal edge, is perpendicular or substantially perpendicular to the main plane of said element, while the other face is inclined.

The constant thickness is intended to overcome the disadvantages of a wedge shape and, in conjunction with the notches, to prevent ejection, thus eliminating the need for screws.

However, in spite of this, it was noted that an expulsion could occur.

### BRIEF SUMMARY OF THE INVENTION

The purpose of the present invention is to propose a system for performing posterior spinal arthrodesis at a facet joint, comprising an implant device and its retaining means, enabling expulsion to be avoided, without utilizing screws to fasten said implant device in the bone material, while also optimizing its retention.

The system for performing posterior spinal arthrodesis at a facet joint comprising an implant device and its retaining means according to the invention, is characterized in that said implant device is in the form of an element comprising, for receiving a bone graft, at least one opening or indentation open on the proximal side and closed on the distal side, and intended to be introduced into a cavity previously made at the location of the facet joint to be immobilized, the opposite faces of said element, comprising reliefs, and in that said retaining means is in the form of a transfacet screw enabling said implant to be compressed in said cavity, i.e. it comprises a threaded distal part of transverse dimensions enabling it to pass into said opening or indentation, so as to enable said compression screw to pass into same in order to be screwed at least into the bone material downstream of said implant, while its proximal part is shaped to bear against or into the bone material upstream of said implant.

Once the implant has been inserted into the cavity previously created at the location of the facet joint to be immobilized, the apophyses are drilled through the implant opening or notch, and the compression screw is then screwed in, trapped in the opening or behind the distal closure of the indentation, thus preventing movement in the direction of expulsion.

An additional feature of the system according to the invention is that the implant device takes the form of a flat element of constant thickness.

In a variant of the system according to the invention, the implant device has a curved shape.

Such a shape is intended to adapt to the curvature of the facet joint, without the need for significant rectification of the interface space.

According to a further feature of the system according to the invention, the implant device has flexibility characteristics.

This feature allows the implant device to adapt to the curvature of the facet joint, whether or not it is curved.

According to a further feature of the system according to the invention, the reliefs on the opposite faces of the implant device consist of notches arranged perpendicularly to the direction of insertion, each of the notches having a two-sided tooth profile, one of which, the one facing the proximal edge, is perpendicular or substantially perpendicular to the main plane of said implant device.

According to a further feature of the system according to the invention, the implant device has a single opening, giving it a substantially annular shape.

In one variant of the system described in this invention, the implant device is U-shaped, delimiting an indentation between its two arms, with the transverse portion connecting the two arms positioned on the distal insertion side.

According to a first particular embodiment of the system according to the invention, the screw has a non-constant thread, and more particularly this pitch is smaller at the proximal part at the distal part, each in one of the articular apophyses to be connected.

In addition to retaining the implant, the compression screw brings the two apophyses together and clamps the implant, thus increasing its primary anchorage and promoting bone fusion.

Advantageously, such a screw has no head, and can be buried completely in the bone material.

According to a further feature of the first particular embodiment of the system according to the invention, the screw comprises a smooth section between the two proximal and distal parts.

According to a further feature of the first particular embodiment of the system according to the invention, the proximal part of the screw has a larger diameter than the distal part.

According to a further feature of the first second particular embodiment of the system according to the invention, the threads of the proximal and distal portions of the screw each comprise a tapping notch.

In a second particular embodiment of the system, the proximal part of the screw consists of a head capable of bearing against the bone material, while the distal part is screwed into the bone material downstream of the implant.

According to a further feature of the second particular embodiment of the system according to the invention, the compression screw has a smooth section below the head, at its part intended to pass through the bone material upstream of the implant.

The smooth section facilitates compression, thanks to the traction created by screwing the threaded part into the bone material downstream of the implant.

It should be noted that the distal part can be entirely threaded, in which case compression requires that the hole made in the bone material upstream of the implant has a diameter greater than that of the threaded distal part and less than that of the head.

### BRIEF DESCRIPTION OF THE SEVERAL VIEWS OF THE DRAWINGS

The advantages and features of the system according to the invention will emerge more clearly from the description which follows and which relates to the appended drawings, which show non-limiting embodiments thereof.

FIG. 1 shows a perspective view of a first embodiment of the system according to the invention.

FIG. 2 shows a schematic cross-sectional view of the same system installed between two apophyses.

FIG. 3 shows a schematic cross-sectional view of the same system from a different angle, along arrow A in FIG. 2.

FIG. 4 shows a schematic cross-sectional view of a second embodiment of the system according to the invention.

### DETAILED DESCRIPTION OF THE INVENTION

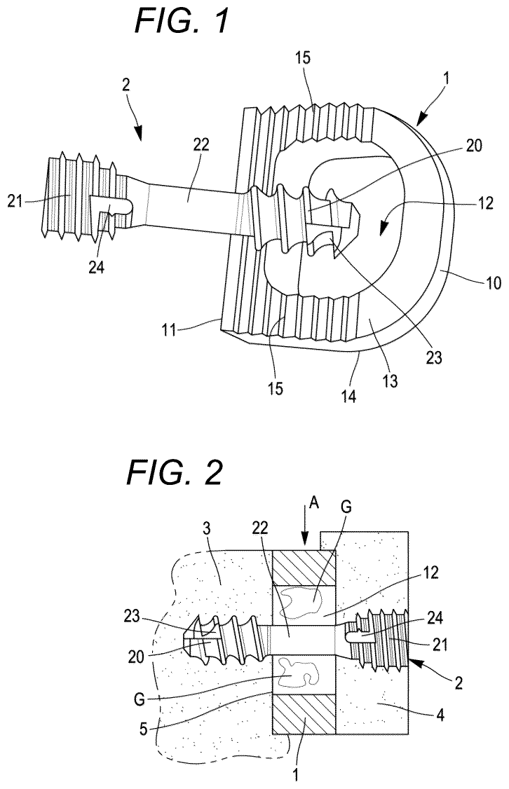

With reference to FIG. 1, it can be seen that the system according to the invention comprises an implant device 1 and a screw 2.

The implant device 1 shown consists of, but is not limited to, an implant device as described in WO 2019016341, i.e. in the form of a substantially flat, D-shaped element intended to be inserted between two apophyses to replace the joint capsule. It thus comprises a distal, rounded insertion side 10 and a proximal, rectilinear side 11, as well as a central opening 12 which gives it a generally annular shape.

Furthermore, each of its two faces 13 and 14, of which only the face 13 is visible in this FIG. 1, is fitted with notches 15 aligned transversely to the insertion axis, and intended to provide an anchorage in the bone material.

According to a first embodiment, the screw 2 consists of a compression screw, which for this purpose has a thread of non-constant pitch, i.e. it comprises a threaded distal part 20 and a threaded proximal part 21, separated by a smooth section 22, while the thread pitch of the proximal part 21 is smaller than the thread pitch of the distal part 20. Furthermore, it can be seen that the diameter of the screw 2 is smaller than the dimensions of the opening 12.

Note that the smooth section 22 is optional, and can be replaced by a transitional threaded zone, moving from one pitch to another.

It should also be noted that the threads of the distal part 20 and proximal part 21 are preferably provided with tapping notches, 23 and 24 respectively. On the other hand, the proximal part 21 has a larger diameter than the distal part 20, so that its thread cuts a different thread in the bone material than the one cut upstream by the thread of the distal part 20.

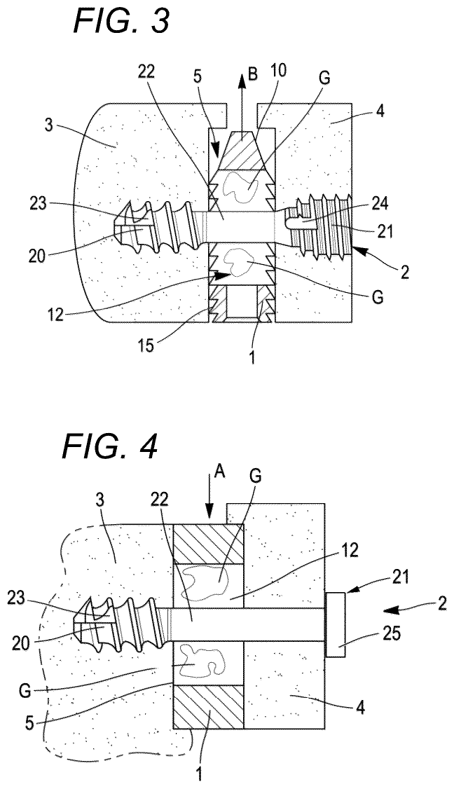

Referring now to FIGS. 2 and 3, we can see the system in place according to the invention, at one of the two facet joints of two vertebrae, and more particularly, seen from the rear, the joint between the left apophysis 3 of one vertebra and the left apophysis 4 of the adjacent vertebra positioned below. The right-hand side is treated in the same way.

In previous steps not shown, the two apophyses 3 and 4 are prepared, i.e. the joint capsule and the osteophytes are removed, and the facet joint space is opened to create a cavity 5 in the interface space, which is then grated down to the cancellous bone in preparation for subsequent fusion.

In FIGS. 2 and 3, the implant 1 has been inserted into the cavity 5 in the direction of the arrow B, and fits snugly into place.

It should be noted that, from an operative point of view, the insertion of the implant 1 on the right side is performed simultaneously with the left side, bearing in mind that in the case of successive insertions, the first insertion would tend to close the interface space on the other side.

It should also be noted that a bone graft G will have been placed in the opening 12 prior to insertion.

It can also be seen in these Figures that the placement of the implant 1 is supplemented by that of the screw 2, a hole having first been drilled in the lower apophysis 4 and then in the upper apophysis 3, passing through the opening 12 of the implant 1, possibly with an aiming device.

The screw 2 is then screwed into this hole, which not only prevents the expulsion of the implant 1, but also, due to the different thread pitches of the distal 20 and proximal 21 parts, enables the apophyses 3 and 4 to be tightened onto the implant 1, and more particularly the notches 15 thereof, thus ensuring perfect retention and promoting bone fusion.

In FIGS. 2 and 3, the screw 2 is perfectly perpendicular to the main plane of the implant 1. However, this is a special case, since the axis of insertion of the screw 2 can be perfectly at an angle to this plane. As drilling is carried out blindly after insertion of the implant 1, the opening 12 is preferably, but not exclusively, large in relation to the transverse dimensions of the screw 2, so as to leave plenty of latitude when an aiming device is not used.

It should also be noted that, insofar as a high-performance aiming device can be used, it may be envisaged that the opening 12 can be tapped, so as to enable the distal part 20 to be screwed in, the thread of which is then extended and the smooth section 22 totally or partially replaced.

Referring now to FIG. 4, a second embodiment of the system according to the invention can be seen.

This differs from the first in that the proximal part 21 of the screw 2 consists of a head 25 capable of bearing against the apophysis 4, while the distal part 20 is threaded and connected to the head 25 by a smooth section 22 intended to be placed in the apophysis 4, so as to be able to achieve compression, i.e. the tightening of the apophyses 3 and 4 on the implant 1.

Note that the head 25 can be milled to embed it in the bone material.

1. A system for performing posterior spinal arthrodesis at a facet joint, the system comprising:
an implant device; and
a retaining means,
wherein said implant device comprises an element for receiving a bone graft having at least one opening or indentation open on the proximal side and closed on the distal side, so as to be introduced into a cavity previously made at the location of the facet joint to be immobilized, and reliefs in the opposite faces,
wherein said retaining means is comprised of a transfacet screw enabling said implant to be compressed in said cavity, and
wherein the screw comprises a threaded distal part of transverse dimensions enabling it to pass into said opening or indentation, so as to enable said screw to pass into same in order to be screwed at least into the bone material downstream of said implant, and a proximal part shaped to bear against or into the bone material upstream of said implant.

2. The system, according to claim 1, wherein said implant device is comprised of a flat element of constant thickness.

3. The system, according to claim 1, wherein said implant device has a curved shape.

4. The system, according to claim 1, wherein said implant device has flexibility characteristics.

5. The system, according to claim 1, wherein the reliefs on the opposite faces are comprised of notches arranged perpendicularly to the direction of insertion, each of the notches having a tooth profile with two sides, one of which, the one facing the proximal edge, is perpendicular to the main plane of said implant device.

6. The system, according to claim 1, wherein said implant device has a single opening so as to form an annular shape.

7. The system, according to claim 1, wherein said implant device is U-shaped, delimiting an indentation between its two arms, with the transverse portion connecting the two arms positioned on the distal insertion side.

8. The system, according to claim 1, wherein the screw has a non-constant thread, and wherein the screw has a pitch smaller at the proximal part than at the distal part, each of said proximal and distal parts so as to be screwed into one of the joint apophyses to be connected.

9. The system, according to claim 8, wherein the screw comprises a smooth section between the two proximal and distal parts.

10. The system, according to claim 8, wherein the proximal part of the screw has a larger diameter than the distal part.

11. The system, according to claim 8, wherein the threads of the distal and proximal parts of the screw, each comprise a tapping notch.

12. The system, according to claim 1, wherein the proximal part of the screw is comprised of a head bearing against the bone material, while the distal part is screwed into the bone material downstream of the implant.

13. The system, according to claim 12, wherein the screw has a smooth section below the head, at its part intended to pass through the bone material upstream of the implant.
